(19) United States
(12) Patent Application Publication
(10) Pub. No.: US 2018/0303486 A1
(43) Pub. Date: Oct. 25, 2018

(54) Embolic Occlusion Device And Method

(71) Applicant: Sequent Medical, Inc., Aliso Viejo, CA (US)

(72) Inventors: Robert Rosenbluth, Laguna Niguel, CA (US); Brian J. Cox, Laguna Niguel, CA (US); William R. Patterson, Aliso Viejo, CA (US)

(73) Assignee: Sequent Medical, Inc., Aliso Viejo, CA (US)

(21) Appl. No.: 15/821,343

(22) Filed: Nov. 22, 2017

Related U.S. Application Data
(63) Continuation of application No. 14/271,099, filed on May 6, 2014, now abandoned.
(60) Provisional application No. 61/819,983, filed on May 6, 2013.

(51) Int. Cl. A61B 17/12; A61B 17/00

(52) U.S. Cl. CPC A61B 17/1214; A61B 17/12163; A61B 17/12168; A61B 2017/00526; A61B 17/12154; A61B 17/1215; A61B 17/12113

(57) ABSTRACT

An occlusion device including a tubular braided member having a first end and a second end and extending along a longitudinal axis, the tubular braided member having a repeating pattern of larger diameter portions and smaller diameter portions arrayed along the longitudinal axis, and at least one metallic coil member extending coaxially along at least a portion of the braided member, the at least one metallic coil member having an outer diameter and an inner diameter, wherein the smaller diameter portions of the tubular braided member have an outer diameter and an inner diameter, and wherein at least one of the outer diameter and inner diameter of the tubular braided member is configured to closely match a directly opposing diameter of the metallic coil member.

### CROSS REFERENCE TO RELATED APPLICATIONS

This patent application is a continuation of U.S. patent Ser. No. 14/271,099 filed May 6, 2014 entitled Embolic Occlusion Device And Method,” claims the benefit under 35 U.S.C. § 119 of U.S. Provisional Application Ser. No. 61/819,983 filed on May 6, 2013 entitled Embolic Occlusion Device And Method,” both of which are incorporated herein in their entireties.

### BACKGROUND

The occlusion of body cavities, blood vessels, and other lumina by embolization is desired in a number of clinical situations, such as, for example, the occlusion of fallopian tubes for the purposes of sterilization, and the occlusive repair of cardiac defects, such as a patent foramen ovale (PFO), patent ductusarteriosis (PDA), left atrial appendage (LAA), and atrial septal defects (ASD). The function of an occlusion device in such situations is to substantially block or inhibit the flow of bodily fluids into or through the cavity, lumen, vessel, space, or defect for the therapeutic benefit of the patient.

The embolization of blood vessels is also desired in a number of clinical situations. For example, vascular embolization has been used to control vascular bleeding, to occlude the blood supply to tumors, and to occlude vascular aneurysms, particularly intracranial aneurysms. Intracranial or brain aneurysms can burst with resulting cranial hemorrhaging, vasospasm, and possibly death. In recent years, vascular embolization for the treatment of aneurysms has received much attention. In such applications, an embolizing device is delivered to a treatment site intravascularly via a delivery catheter (commonly referred to as a “microcatheter”). Several different treatment modalities have been shown in the prior art. One approach that has shown promise is the use of embolizing devices in the form of microcoils. These microcoils may be made of biocompatible metal alloy(s) (typically a radiopaque material such as platinum or tungsten) or a suitable polymer.

A specific type of microcoil that has achieved a measure of success is the Guglielmi Detachable Coil (“GDC”), described in U.S. Pat. No. 5,122,136 to Guglielmi at al. The GDC employs a platinum wire coil fixed to a stainless steel delivery wire by a solder connection. After the coil is placed inside aneurysm, an electrical current is applied to the delivery wire, which electrolytically disintegrates the solder junction, thereby detaching the coil from the delivery wire. The application of current also creates a positive electrical charge on the coil, which attracts negatively-charged blood cells, platelets, and fibrinogen, thereby potentially increasing the thrombogenicity of the coil. Several coils of different diameters and lengths can be packed into an aneurysm until the aneurysm is completely filled. The coils thus create a thrombus and hold the thrombus within the aneurysm, inhibiting the displacement and fragmentation of the thrombus. A limitation of embolic coils is that they can only fill up to about 35% of the volume of an intracranial aneurysm due at least partially to early blockage of the opening or neck of the aneurysm, thus inhibiting the passage of subsequent coils. With the remaining space unfilled, a clot that forms due to the thrombosis can have flow channels and/or fibrin turnover, resulting in an unstable clot. Instability can promote compaction of the coil and clot embolus, leading to the need for retreatment. Higher volume devices using larger coil diameters or attached hydro gels have been tried, but their increased size and different characteristics can complicate their delivery, thus inhibiting their widespread use.

Alternative vasa-occlusive devices are exemplified in U.S. patent application Ser. No. 12/434,465, published as U.S. Pat. App. Pub. No. 2009/0275974 to Marchand et al., entitled “Filamentary Devices for Treatment of Vascular Defects”, and filed May 1, 2009, Ser. No. 12/939,901, published as U.S. Pat. App. Pub. No. 2011/0152993 to Marchand et al., entitled “Multiple Layer Filamentary Devices for Treatment of Vascular Defects”, and filed Nov. 4, 2010 and Ser. No. 13/439,754, published as U.S. Pat. App. Pub. No. 2012/0197283 to Marchand et al., entitled “Multiple Layer Filamentary Devices for Treatment of Vascular Defects”, and filed Apr. 4, 2012; and U.S. patent application Ser. No. 13/464,743, published as U.S. Pat. App. Pub. No. 2012/0283768 to Cox et al., entitled “Method and Apparatus for the Treatment of Large and Giant Vascular Defects”, and filed May 4, 2012; all of which are assigned to the assignee of the subject matter of the present disclosure, and are incorporated by reference.

### SUMMARY

The present disclosure provides for an occlusion device including a tubular braided member having a first end and a second end and extending along a longitudinal axis, the tubular braided member having a repeating pattern of larger diameter portions and smaller diameter portions arrayed along the longitudinal axis, and at least one metallic coil member extending coaxially along at least a portion of the braided member, the at least one metallic coil member having an outer diameter and an inner diameter, wherein the smaller diameter portions of the tubular braided member have an outer diameter and an inner diameter, and wherein at least one of the outer diameter and inner diameter of the tubular braided member is configured to closely match a directly opposing diameter of the metallic coil member.

The present disclosure additionally provides for an embolic occlusion device including an expandable braided element extending along a longitudinal axis between a first end and a second end, the braided element being configured as a series of portions having a first diameter alternating with portions having a second diameter larger than the first diameter arrayed along the longitudinal axis, and a metallic coil element having an outside diameter smaller than the second diameter and disposed coaxially with a portion of the braided element having the first diameter.

The present disclosure additionally provides for an embolic occlusion device, including an expandable braided element extending along a longitudinal axis between a first end and a second end, the braided element being configured as a series of portions having a first diameter alternating with portions having a second diameter larger than the first diameter arrayed along the longitudinal axis, and a plurality of metallic coil elements, each having an outside diameter smaller than the second diameter and an inside diameter conforming to the first diameter, each of the metallic coil elements being disposed coaxially around one of the portions of the braided element having the first diameter.

### BRIEF DESCRIPTION OF THE DRAWINGS

FIG. 1 is a simplified view of a delivery catheter placed within an aneurysm, for delivery of an occlusion device in accordance with present disclosure.

FIG. 2 is an elevation view of an occlusion device according to an embodiment of the present disclosure.

FIG. 3A is an elevation view of a braided member according to an embodiment of the present disclosure.

FIG. 3B is a detailed view of the braided filaments of a braided member of the type shown in FIG. 3A.

FIG. 4A is an elevation view of an occlusion device according to an embodiment of the present disclosure.

FIG. 4B is an elevation view of an occlusion device according to an embodiment of the present disclosure.

FIG. 4C is an elevation view of an occlusion device according to an embodiment of the present disclosure.

FIG. 5 is an elevation view of an occlusion device according to an embodiment of the present disclosure.

FIG. 6 is a partially sectional view of an occlusion device coupled to a delivery device according to an embodiment of the present disclosure, disposed within the lumen of a delivery catheter.

FIG. 7 is an elevation view of an occlusion device having a secondary coiled or helical configuration according to an embodiment of the present disclosure.

FIG. 8 is a view of an occlusion device in accordance with an embodiment of the present invention being delivered into an aneurysm.

### DETAILED DESCRIPTION

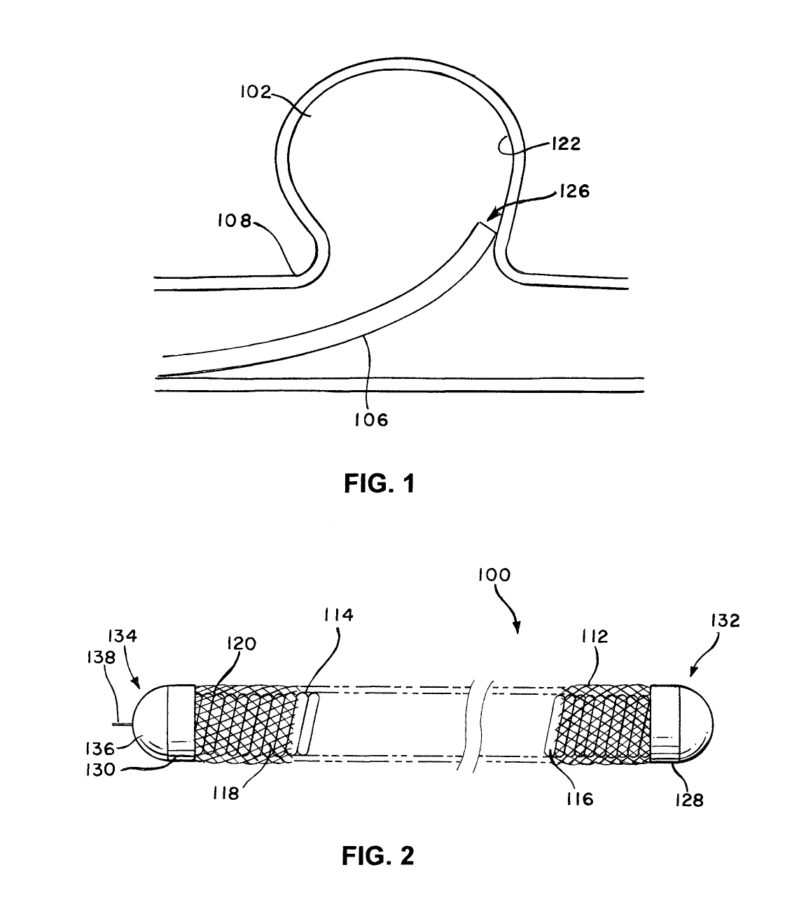

The embodiments of the present disclosure provide for more advanced and improved occlusion devices, for example an occlusion device in the form of an elongate, expandable embolic device 100 (FIG. 2). The elongate, expandable embolic device 100 exhibits excellent stability after deployment in a target site 102 (e.g., an aneurysm, as shown in FIG. 1) that has formed from a blood vessel wall 108. The elongate, expandable embolic device 100, as well as other embodiments of an occlusion device in accordance with the present disclosure, has improved space filling ability within a target site 102, and a wider application in target sites 102 of varying sizes, as compared to conventional occlusion devices. The elongate, expandable embolic device 100 and other embodiments also have increased efficiency for treating and occluding target sites 102. The elongate, expandable embolic device 100 is configured to be delivered through a delivery catheter 106, for example a microcatheter, having an inner lumen internal diameter of 0.033 inches or less, or 0.021 inches or less, or even 0.017 inches or less.

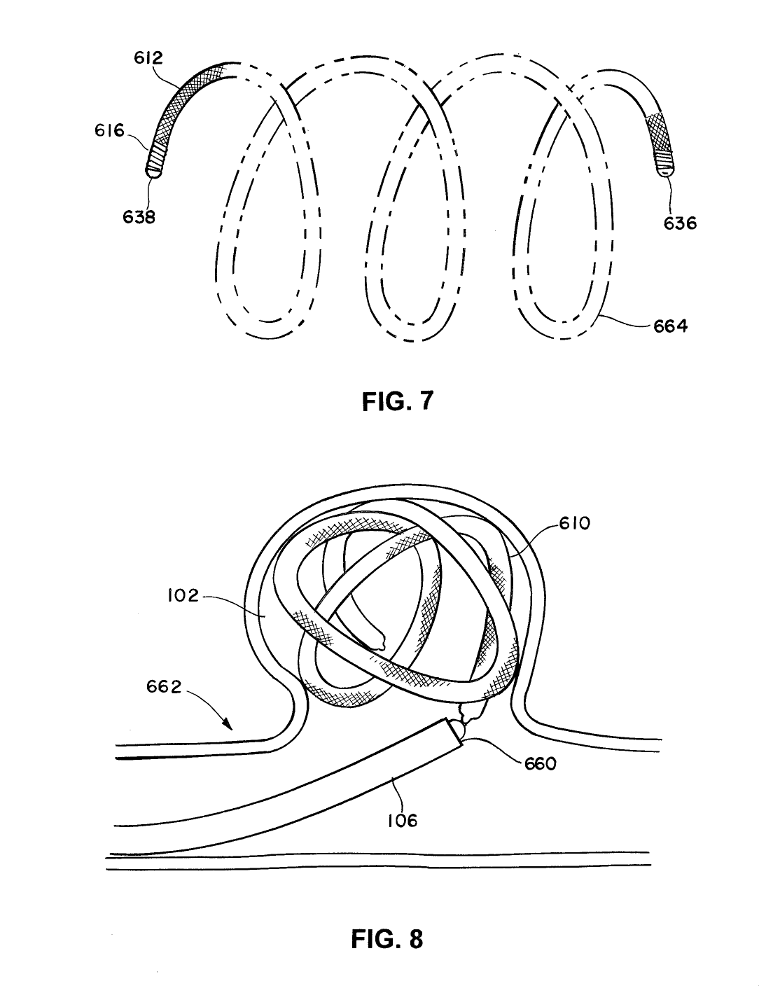

In the embodiment of FIG. 2, the elongate, expandable embolic device 100 comprises an expandable braided outer member 112 and a flexible, elongate inner member 114, preferably comprising one or more coil elements 116, that serves as a core or backbone of the embolic device 100 shown in FIG. 2. In some embodiments, the embolic device 100 comprises one or more coil elements 116 having a preset helical configuration (see FIG. 7), wherein the expandable outer member 112 is connected to, or in a co-axial arrangement around, at least a portion of the inner member 114. The outer member 112, which may advantageously comprise an expandable mesh portion 120, is shown in a collapsed state in FIG. 2, in which it allows the embolic device to be passed, by a delivery device or pusher (described below), through the delivery catheter 106 (see FIG. 1) until the embolic device is delivered into the target site 102 through the distal end 126 of the delivery catheter 106. After embolic device is thus deployed into the target site 102, it is detached from the delivery device or pusher, whereupon expansion of the mesh portion 120 causes the braided outer member 112 to assume an expanded state. When the outer member 112 is in its expanded state, the mesh portion 120 of the outer member 112 may inhibit movement within the target site 102, and it may also inhibit dislodgement and potential downstream embolization of the embolic device 100. The outer member 112 may provide substantially more volumetric filling by forming at least one substantially closed volume (other than the pores or openings in the mesh portion 120) with substantially more surface area for thrombus formation, and thus more efficient thrombosis and embolization of the target site 102. An expandable mesh portion 120 that is formed of a large number of relatively fine (small gauge) wires 118 may also provide better grip or fixation against an inner wall 122 of the target site 102 (see FIG. 1) or other tissue, and thus provide an implant with improved stability. The wires 118 of the braided outer member 112 may be secured together at either the distal end 132 or the proximal end 134 of the embolic device 100, and preferably at both ends, by a distal end hub 128 and/or a proximal end hub 130, either or both of which may comprise radiopaque marker bands, for example comprising platinum. The proximal end 134 may include a detachable coupling element 136, for example, a tether 138, to which the embolic device is detachably coupled to a delivery device or pusher (see below). After the embolic device 100 is positioned within the target site 102 and deployed from the distal end 126 of the delivery catheter 106, the coupling element 136 may be controllably broken, melted, or otherwise severed from the delivery device or pusher, as described below.

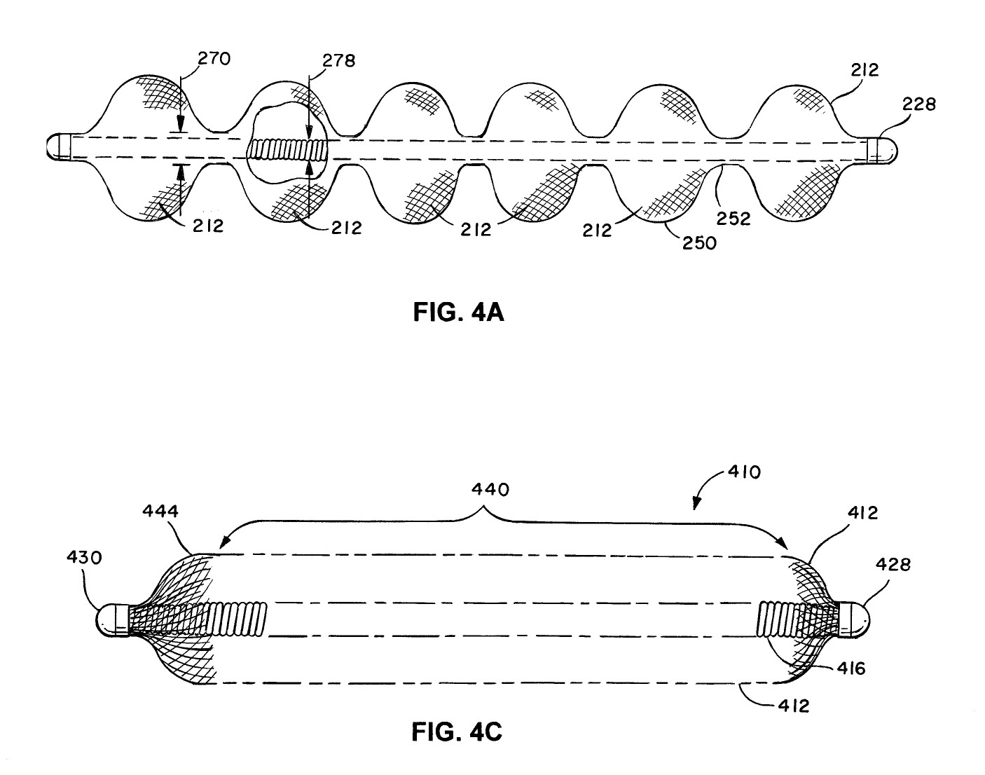

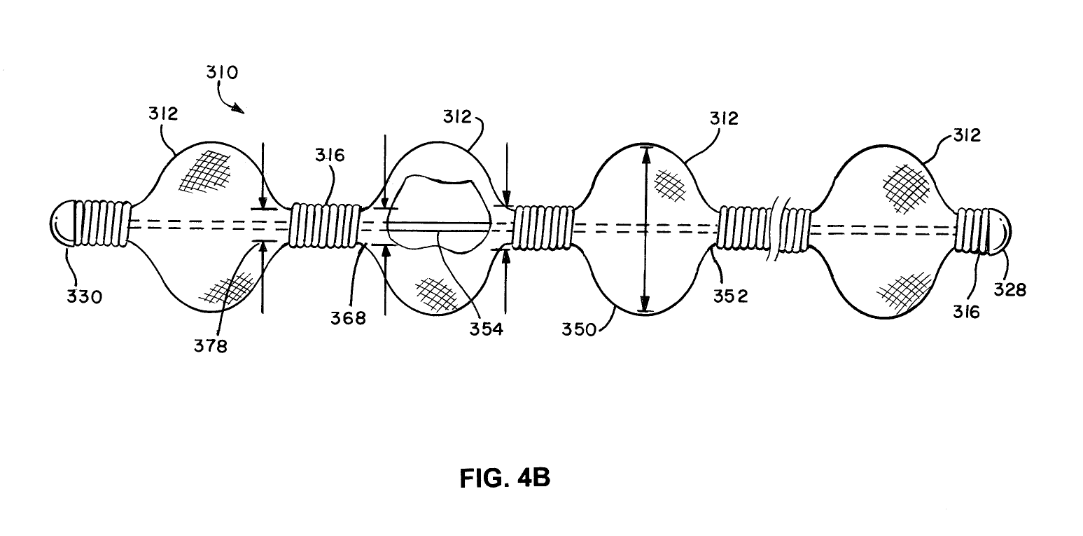

Several embodiments of occlusion devices 210, 310, 410 are shown in FIGS. 4A, 4B, and 4C. As shown in FIG. 4C, a braided outer member 412 may comprise a continuous expandable covering 440 extending along a longitudinal axis and tapering down at a first end 442 and a second end 444, to which it may be secured to an inner axial coil member 416 with a first end hub 428 and a second end hub 430. Alternatively, as shown in FIGS. 4A and 4B, all or a portion of an expandable braided member 212, 312 may have an undulating or wave-like configuration extending along a longitudinal axis and comprising increased diameter portions 250, 350 alternating with decreased diameter portions 252, 352. The braided members 212, 312 may be secured to an axial coil member 216, 316 at either end by first end hubs 228, 328 and second end hubs 230, 330. The occlusion device 210 of FIG. 4A comprises one or more inner axial coil members 216 that are completely internal to the braided member 212. The occlusion device 310 of FIG. 4B comprises one or more axial coil members 316 that wind around the decreased diameter portions 352 of the braided member 312.

For tensile integrity of any of the occlusion devices 210, 310, 410, a stretch resistant thread or filament 354 (FIG. 4B) may extend axially through the occlusion device and be secured at each end of the occlusion device 210, 310, 410. Exemplary materials for the filament 354 may include, but not be limited by: polymers such as polyolefin, polyolefin elastomer, polyethylene, ultra-high molecular weight polyethylene such as Spectra® or Dyneema®, polyester (PET), polyamide (Nylon), polyurethane, polypropylene, block copolymers such as PEBAX or the thermoplastic polyester marketed by E. I. DuPont de Nemours under the trademark Hytrel®, ethylene vinyl alcohol (EVA), or rubbery materials such as silicone, latex, and similar flexible polymers such as those produced by Kraton Polymers U.S., LLC, of Houston, Tex. A particularly useful material for the tether is Paramyd®, which is a para-aramid (poly-paraphenyleneterepthalamide) and is commercially available from Aramid, Ltd., Hilton Head, S.C. In some cases, the polymer may also be cross-linked by radiation to manipulate its tensile strength and melt temperature. Other materials that may be useful for tether construction include wholly aromatic polyester polymers which are liquid crystal polymers (LCP) that may provide high performance properties and are highly inert. A commercially available LCP polymer is Vectran, which is produced by Kuraray Co. (Tokyo, Japan). The selection of the material may depend on the melting or softening temperature, the power used for detachment, and the body treatment site. The tether 138 may be joined to the occlusion device 210, 310, 410 by crimping, welding, knot tying, soldering, adhesive bonding, or other means known in the art. In all occlusion devices 110, 210, 310, 410, the coil members 116, 216, 316, 416 may be formed from radiopaque (e.g. platinum) wire, to provide radiopacity along all or a portion of the length. In addition, the wires of the braided members 112, 212, 312, 412 may include some platinum wires or drawn filled tubes (DFT) having platinum cores (or other radiopaque material), in order to enhance the radiopacity of the braided members 112, 212, 312, 412.

The coil members 216, 316 in the embodiments of FIGS. 4A and 4B provide increased axial pushability to the occlusion device 210, 310. In the occlusion device 210 of FIG. 4A, the inner diameter 270 of the braided member 212 at the decreased diameter portions 252 is configured to closely match and/or conform to the outer diameter 278 of the metallic coil member 216. For example, the inner diameter 270 may be made or formed approximately equal to the outer diameter 278. In the occlusion device 310 of FIG. 4B, the outer diameter 368 of the braided member 312 at the decreased diameter portions 352 is configured to closely match and/or conform to the inner diameter 378 of the metallic coil member 316. For example, the outer diameter 368 may be made or maintained approximately equal to the inner diameter 378. Additionally, the undulating or wave-like configuration of the alternating increased diameter portions 250, 350 and decreased diameter portions 252, 352 allows for flexibility, particularly in enabling the occlusion device 210, 310 to take a secondary shape within a vascular defect.

In some embodiments, the braided members may form discs or globular shapes. In FIG. 4C, the generally cylindrical braided member 412 may have an expanded diameter that is substantially larger than the diameter of standard embolic coils. In some embodiments, the diameter of the braided member may be between about 0.5 mm and 5.0 mm and in other embodiments between about 1.0 mm and 3.0 mm. The coil member(s) 416 may be included within the ends, for example, attached to the end hubs 428, 430, and may even extend beyond the braided member 412.

In some embodiments, the total surface area, defined as the surface area of all the filamentary elements that comprise the braided member(s) 112, 212, 312, 412 of the occlusion device 110, 210, 310, 410 may be between about two times and about fifty times the total surface area of a similar length standard helical embolic coil. Further, a standard embolic coil has an even lower effective surface area, as only the outer surface is in contact with flowing blood. Thus, the effective surface area of a conventional embolic coil is not substantially greater than the surface area of the cylinder formed by the primary wind of the coil. The inner surface of the coil is generally only in contact with blood that seeps into the coil and not with flowing blood. Thus, the effective surface area of a conventional embolic coil would be only marginally greater than its external surface area. The external surface area may be approximated by the surface area equation for a cylinder where the radius is the radius of the primary wind of the coil. In some embodiments, the total effective surface area of the occlusion device 110, 210, 310, 410, defined as the total surface area of all filaments that come into contact with flowing blood, may be between about ten times and about one hundred times that of a similar length conventional embolic coil. The surface area of a cylinder may be calculated by:

Surface of the cylinder=2nr×L
Where r is the radius, and
L is the length

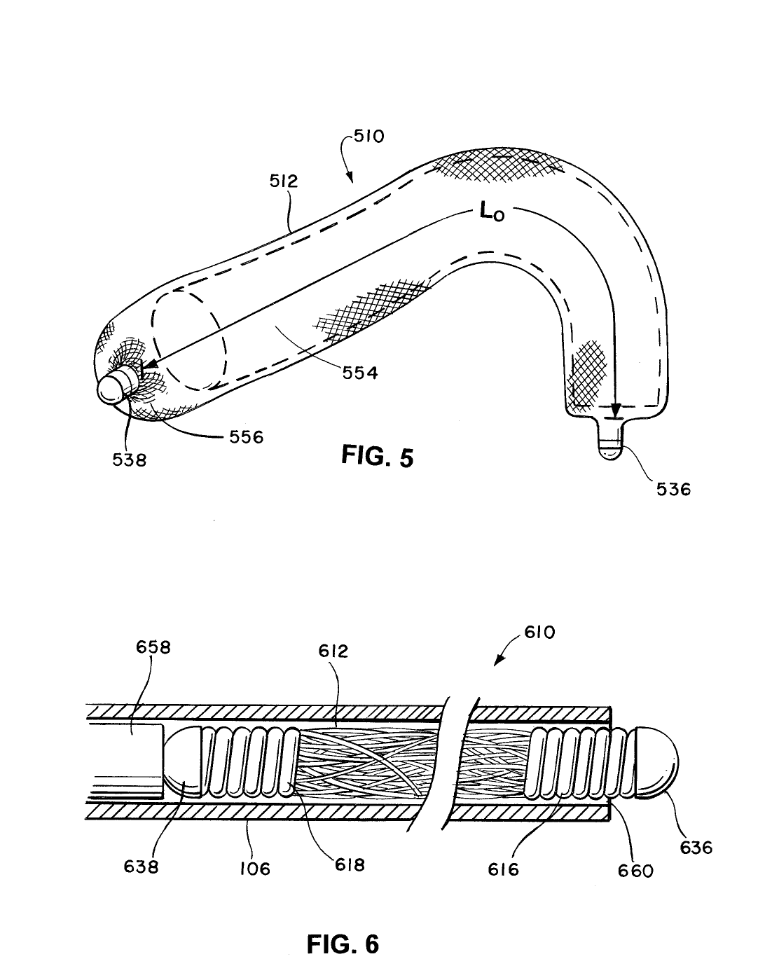

In some embodiments, the braided member 112, 212, 312, 412 may form a substantially closed volume (other than the pores of the braid). In some embodiments, such as the braided member 512 of the occlusion device 510 of FIG. 5, the closed volume(s) may define a cylindrical space 554, with a volume V_{c} that is a function of the total length L0 of the occlusion device 510. For example, in some embodiments, the closed volume Vc may be about 0.5Lₒ and about 6.0 Lₒ, and in some embodiments between about 2.0 Lₒ and about 4.0 Lₒ. FIG. 5 further illustrates that the filaments 556 of the braided member 512 may be secured at either end by end hubs 536, 538.

FIG. 6 illustrates a radially expandable embolic device 610 in accordance with an exemplary embodiment of this disclosure. The embolic device 610 includes a radially expandable portion 612 that is formed of a braided fiber or wire mesh. The expandable portion 612 may advantageously be disposed between a distal coil portion 616 and a proximal coil portion 618 that provide needed axial rigidity. The expandable portion 612 has a relaxed, expanded state from which it may be radially compressed to provide a radially compressed or collapsed configuration for the device 610. Releasing the expandable portion 612 from a compressive force allows it resiliently to expand to its relaxed state, thereby giving the device 610 a radially expanded configuration. The distal coil portion 616 is secured to a distal end cap or hub 636, and the proximal coil portion 618 is secured to a proximal end cap or hub 638.

Alternatively, the embolic device 610 may have a unitary coil forming an axial inner core between the end caps 636, 638, and the expandable braided mesh portion 612 may form a coaxial outer element disposed around the coil and likewise secured to the end caps 636, 638. In either case, the embolic device 610 is detachably connected to the distal end of a delivery device or pusher 658 by means such as a severable tether 138 (FIG. 2) fixed to the proximal end cap 638. It is understood that an expandable embolic device in accordance with any of the previously described embodiments may similarly be detachably connected to the distal end of the pusher 658.

As illustrated in FIGS. 1, 6, 7, and 8, the subject matter of the present disclosure provides methods for occluding a body cavity or vascular defect 102. Embodiments of such methods include inserting a delivery catheter 106 (e.g., a microcatheter) through the vasculature until its distal end 126 enters a target site 102; using a pusher (e.g., the pusher 658 shown in FIG. 6) to pass an expandable occlusion device (e.g., the expandable embolic device 610 shown in FIG. 6), detachably connected to the distal end of the pusher 658, through the delivery catheter 106 while in a radially collapsed configuration until the embolic device 610 emerges from the distal end 126 of the delivery catheter 106 (FIG. 8) and enters the target site 102, wherein the occlusion device 610 forms a looping, helical, or arcuate secondary form 664 (as shown in FIG. 7); allowing the embolic device 610, once free of the distal end 126 of the delivery catheter 106, to assume a radially expanded configuration (FIG. 8), thereby forming at least one substantially closed volume (other than the mesh openings of the braided mesh portion 612); detaching the embolic device 610 from the pusher 658; and withdrawing the delivery device or pusher 658 from the target site 102 and the vasculature 662 (FIG. 8). The delivery catheter 106 may either be withdrawn along with, or separately from, the pusher 658, or it may be left in place with its distal end 126 in the target site 102 if it is desired to deploy a second embolic device in the target site.

In any of the embodiments described herein, the expandable braided member 112, 212, 312, 412, 512, 612 can be a braid of wires, filaments, threads, sutures, fibers or the like, that have been configured to form a fabric or structure having openings (e.g., a porous fabric or structure). The braided member 112, 212, 312, 412, 512, 612 and the coil member 116, 216, 316, 416, 516, 616 can be constructed using metals, polymers, composites, and/or biologic materials. Polymer materials can include polyesters, for example Dacron® or polyethylene terephthalate (PET), polypropylene, nylon, Teflon®, PTFE, ePTFE, TFE, TPE, PLA, silicone, polyurethane, polyethylene, ABS, polycarbonate, styrene, polyimide, Polyether block amide, such as PEBAX®, thermoplastic elastomers, such as Hytrel®, poly vinyl chloride, HDPE, LDPE, Polyether ether ketone, such as PEEK, rubber, latex, or other suitable polymers. Other materials known in the art of vascular implants can also be used. Metal materials can include, but are not limited to, nickel-titanium alloys (e.g. Nitinol), platinum, cobalt-chrome alloys, 35N LT®, Elgiloy®, stainless steel, tungsten or titanium. In certain embodiments, metal filaments may be highly polished or surface treated to further improve their hemo-compatibility. In some embodiments, it is desirable that the occlusion device 110, 210, 310, 410, 510, 610 be constructed solely from metallic materials without the inclusion of any polymer materials, i.e. polymer free.

In any of the embodiments described herein, the coil member(s) 116, 216, 316, 416, 516, 616 and/or braided member(s) 112, 212, 312, 412, 512, 612 may be heat-set into a secondary coil (such as the secondary form 664 of FIG. 7) or other arcuate configuration as is known in the art of embolic coils. The secondary configuration may be helical, as in FIG. 7, or a three-dimensional (3-D) shape such as a cone, sphere or ovoid configuration. Various 3-D coil configurations are shown in U.S. Pat. Nos. 6,024,765 and 6,860,893, both to Wallace et al., and herein incorporated in their entirety by reference.

For braided portions, components, or elements, the braiding process can be carried out by automated machine fabrication or can be performed by hand. For some embodiments, the braiding process can be carried out by the braiding apparatus and process described in U.S. Pat. No. 8,261,648, entitled “Braiding Mechanism and Methods of Use” by Marchand et al., which is herein incorporated by reference in its entirety. In some embodiments, a braiding mechanism may be utilized that comprises a disc defining a plane and a circumferential edge, a mandrel extending from a center of the disc and generally perpendicular to the plane of the disc, and a plurality of actuators positioned circumferentially around the edge of the disc. A plurality of filaments are loaded on the mandrel such that each filament extends radially toward the circumferential edge of the disc and each filament contacts the disc at a point of engagement on the circumferential edge, which is spaced apart a discrete distance from adjacent points of engagement. The point at which each filament engages the circumferential edge of the disc is separated by a distance “d” from the points at which each immediately adjacent filament engages the circumferential edge of the disc. The disc and a plurality of catch mechanisms are configured to move relative to one another to rotate a first subset of filaments relative to a second subset of filaments to interweave the filaments. The first subset of the plurality of filaments is engaged by the actuators, and the plurality of actuators is operated to move the engaged filaments in a generally radial direction to a position beyond the circumferential edge of the disc. The disc is then rotated a first direction by a circumferential distance, thereby rotating the second subset of filaments a discrete distance and crossing the filaments of the first subset over the filaments of the second subset. The actuators are operated again to move the first subset of filaments to a radial position on the circumferential edge of the disc, wherein each filament in the first subset is released to engage the circumferential edge of the disc at a circumferential distance from its previous point of engagement. Such a braiding apparatus may allow for the mixing of different wire diameters to a greater extent than is generally achievable with conventional carrier-type braiders. Further, such a braiding mechanism may allow for the braiding of very fine wires with a lower rate of breakage.

The process of fabrication of the occlusion device 110, 210, 310, 410, 510, 610 may comprise a method for braiding filaments to form a tubular medical implant device, comprising the steps of: providing a plurality of filaments, an automated mechanism configured to move the filaments in discrete radial and rotational movements, and weights for attachment to each filament; attaching a plurality of filaments to the mandrel and extending the filaments radially from the mandrel; placing each of the filaments in tension using the weights; operating the braiding mechanism to move the filaments in a series of discrete radial and rotational movements; and, forming a tubular braid about the mandrel.

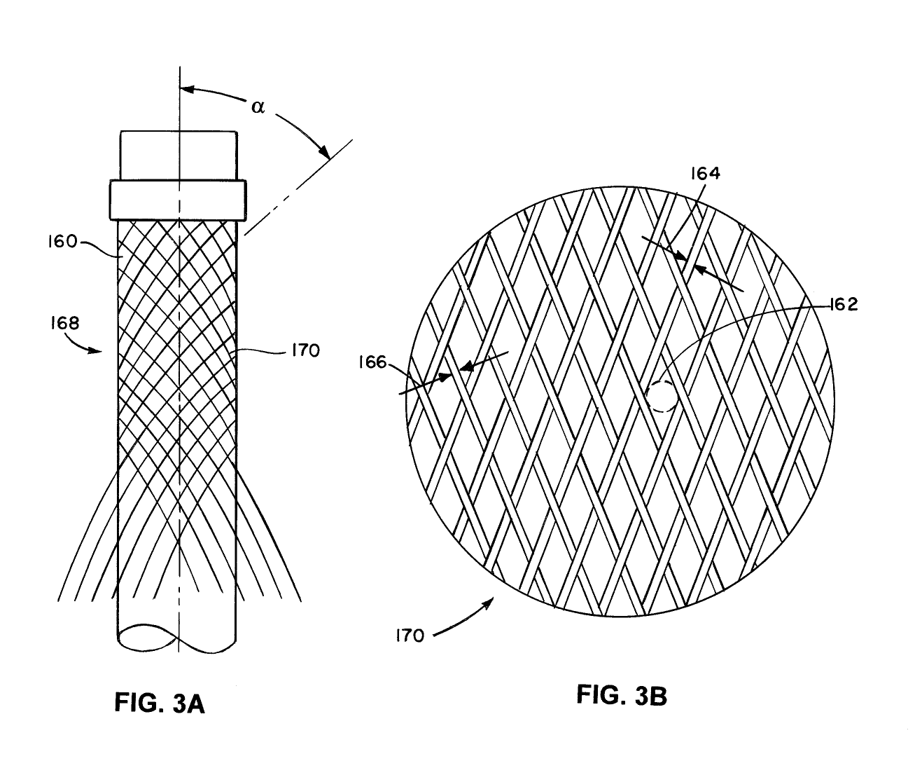

FIG. 3A shows a braided tubular member 168 being formed over a mandrel 160 as is known in the art of tubular braid manufacturing. The braid angle a can be controlled by various means known in the art of filament braiding. The tubular braided mesh 170 can then be further shaped using a heat setting process. Referring to FIG. 3A, as is known in the art of heat-setting a braiding filament, such as Nitinol wires, a fixture, mandrel or mold can be used to hold the braided tubular structure in its desired configuration while subjected to an appropriate heat treatment such that the resilient filaments of the braided tubular member 168 assume or are otherwise shape-set to the outer contour of the mandrel or mold. The filamentary elements of a mesh device or component can be held by a fixture configured to hold the device or component in a desired shape and, in the case of Nitinol wires, heated to about 475° C. to about 525° C. for about 5 to about 30 minutes to shape-set the structure. Such braids of shape memory and/or elastic filaments are herein referred to as “self-expanding.” Other heating processes are possible and will depend on the properties of the material selected for braiding.

In some embodiments, braid filaments of varying diameters may be combined in all or portions of the braided member 112, 212, 312, 412, 512, 612 to impart different characteristics, e.g. stiffness, elasticity, structure, radial force, pore size, embolic filtering ability, and/or other features. For example, in the embodiment shown in FIG. 3B, the braided mesh 170 has a first filament diameter 164 and a second filament diameter 166, smaller than the first filament diameter 164. In some embodiments, the diameter of the braid filaments can be less than about 0.25 millimeters (mm). In other embodiments, the filament diameter may range from about 0.01 mm to about 0.15 mm. In some embodiments, the braided member 112, 212, 312, 412, 512, 612 may be fabricated from wires with diameters ranging from about 0.015 mm to about 0.1 mm. In some embodiments, the braided member 112, 212, 312, 412, 512, 612 may be fabricated from wires with diameters ranging from about 0.025 mm to about 0.06 mm.

As used herein, “pore size” of the braided member 112, 212, 312, 412, 512, 612 refers to the diameter of the largest circle 162 that fits within an individual cell of a braid (see FIG. 3B). The average pore size of the braided member 112, 212, 312, 412, 512, 612, which may be determined by measuring at least five pores and taking the mean, can be less than about 0.5 mm in some embodiments. In some embodiments, the average pore size may be between about 0.1 mm and 0.25 mm. In some embodiments, the pore structure may vary over the expanded braided member 112, 212, 312, 412, 512, 612 such that the largest pores are generally present in the center of the braided member 112, 212, 312, 412, 512, 612. In this case, the average pore size would be measured near the center.

In some embodiments, the braided member 112, 212, 312, 412, 512, 612 filament count is greater than 30 filaments per inch. In one embodiment, the total filament count for the braid is between about 30 and about 280 filaments, in other embodiments between about 60 and about 200 filaments, or in further embodiments between about 48 and about 160 filaments. In some embodiments, the total filament count for the braided member 112, 212, 312, 412, 512, 612 is between about 70 and about 240 filaments.

Since the moment of inertia is a function of filament diameter to the fourth power, a small change in the diameter greatly increases the moment of inertia. Thus, a small change in filament size can have substantial impact on the deflection at a given load and thus the compliance of the device. Thus, the stiffness can be increased by a significant amount without a large increase in the cross-sectional area of a collapsed profile of the device. This may be particularly important as device embodiments are made larger to treat larger sites, organs or defects. As such, some embodiments of devices for treatment of a target site may be formed using a combination of filaments with a number of different diameters such as 2, 3, 4, 5, or more different diameters or transverse dimensions. In device embodiments where filaments with two different diameters are used, some larger filament embodiments may have a transverse dimension of about 0.0015 inches to about 0.005 inches, and some small filament embodiments may have a transverse dimension or diameter of about 0.0006 inches to about 0.0015 inches. The ratio of the number of large filaments to the number of small filaments may be between about 4 and 16 and may also be between about 6 and 10. In some embodiments, the difference in diameter or transverse dimension between the larger and smaller filaments may be less than about 0.003 inches, and in other embodiments, less than about 0.002 inches. In some embodiments, the difference in diameter or transverse dimension between the largest and smallest filaments may be more than about 0.0075 inches, and in other embodiments, more than about 0.0125 inches.

In any of the embodiments described herein, the braided member 112, 212, 312, 412, 512, 612 may comprise two or more layers. For embodiments with a plurality of layers, the inner layer may comprise larger filaments on average or a greater number of large filaments relative to the outer layer(s) and thus be a structural layer that is configured to drive the outer braid layer(s) radially outward. The outer braid layers may be occlusive layers comprising very fine wires, the type of which have not normally been used in occlusive implants. In some embodiments, the average diameter of filaments of an occlusive braid may be less than about 0.001 inches and in some embodiments between about 0.0004 inches and about 0.001 inches.

In some embodiments one or more eluting filament(s) may be interwoven into the braided member 112, 212, 312, 412, 512, 612 to provide for the delivery of drugs, bioactive agents or materials. The interwoven filaments may be woven into the lattice structure after heat treating (as discussed herein) to avoid damage to the interwoven filaments by the heat treatment process. In some embodiments, some or all of the occlusion device may be coated with various polymers or bioactive agents to enhance its performance, fixation and/or biocompatibility. In other embodiments, the device may incorporate cells and/or other biologic material to promote sealing and/or healing.

Embodiments for deployment and release of therapeutic devices, such as deployment of embolic devices or stents within the vasculature of a patient, may include connecting such a device via a releasable connection to a distal portion of a pusher or other delivery apparatus member. For example, the delivery and detachment apparatus 658 in FIG. 6. The therapeutic device may be detachably mounted to the distal portion of the apparatus by a filamentary tether, string, thread, wire, suture, fiber, or the like, which may be referred to above as the tether. For some embodiments, the detachment of the device from the delivery apparatus of the delivery system may be effected by the delivery of energy (e.g. current, heat, radiofrequency (RF), ultrasound, vibration, or laser) to a junction or release mechanism between the device and the delivery apparatus. Once the device has been detached, the delivery system may be withdrawn from the patient's vasculature or body. An exemplary detachment system, described in co-owned U.S. Pat. No. 8,597,323, Plaza et al., entitled “DELIVERY AND DETACHMENT SYSTEMS AND METHODS FOR VASCULAR IMPLANTS,” and which is herein incorporated by reference in its entirety, comprises a delivery pusher apparatus, an implant device that is detachably connected to the delivery pusher apparatus by a tether having a distal end connected to a proximal end of the implant device, wherein the tether is substantially non-tensioned when connecting the implant device to the delivery pusher apparatus. An electrical heating element is configured coaxially around at least a portion of the tether, wherein heat generated by the heating element severs the tether at a point near the proximal end of the implant device. The heating element may comprise an electric coil that includes a plurality of windings, at least one which is wound in a reverse direction over the other windings to form a coil region having two winding layers. The coiled heating element may have between about 2 and about 10 windings in the heat-generating zone.

With regard to the above detailed description, like reference numerals used therein refer to like elements that may have the same or similar dimensions, materials and configurations. While particular forms of embodiments have been illustrated and described, it will be apparent that various modifications can be made without departing from the spirit and scope of the embodiments. Accordingly, it is not intended that the invention be limited by the foregoing detailed description.

1. An embolic occlusion device, comprising:
an expandable element of braided metal filaments extending along a longitudinal axis between a proximal end and a distal end, the expandable element forming an arcuate secondary form; and
a coil extending from the distal end of the expandable element.

2. The device of claim 1, wherein the expandable element is configured as a series of portions having a first diameter alternating with portions having a second diameter larger than the first diameter arrayed long the longitudinal axis wherein the expandable element is configured as a series of portions having a first diameter alternating with portions having a second diameter larger than the first diameter arrayed long the longitudinal axis.

3. The device of claim 1, wherein the metal filaments comprise nitinol.

4. The device of claim 1, wherein the metal filaments comprise shape memory filaments.

5. The device of claim 1, further comprising an end cap coupled to a distal end of the coil.

6. The device of claim 1, further comprising a delivery device having a proximal and distal end, wherein the proximal end of the expandable element is coupled to the distal end of the delivery device.

7. The device of claim 6, wherein the proximal end of the expandable element if coupled to the distal end of the delivery device via a severable tether.

8. The device of claim 7, wherein the delivery device comprises a heating element configured coaxially around at least a portion of the tether, the heating element capable of generating heat to sever the tether.

9. The device of claim 1, wherein the coil includes a secondary shape.

10. The device of claim 9, wherein the secondary shape comprises a helical shape.

11. The device of claim 1, wherein the metal filaments comprise platinum.

12. The device of claim 1, wherein the metal filaments include a first group of filaments having a first diameter and a second group of filaments having a second diameter, different from the first diameter.

13. The device of claim 1, wherein the metal filaments have a diameter between about 0.015 mm and about 0.1 mm.

14. The device of claim 1, whereon the metal filaments have a diameter between about 0.025 mm and about 0.06 mm.

15. The device of claim 1, wherein the braided metal filaments form an average pore size in the expandable element that is less than about 0.5 mm.

16. The device of claim 1, wherein the total number of metal filaments is between about 30 and about 280.

17. The device of claim 1, further comprising a biologic material configured to promote sealing or healing.

18. The device of claim 1, further comprising an elongate stretch resistant member extending longitudinally within the expandable element.

19. The device of claim 1, further comprising a metallic coiled element extending longitudinally within the expandable element.

20. The device of claim 19, therein the metallic coiled element is radiopaque.
